(19) United States
(12) Patent Application Publication
(10) Pub. No.: US 2013/0034857 A1
(43) Pub. Date: Feb. 7, 2013

(54) OPTICAL ANALYSIS APPARATUS AND OPTICAL ANALYSIS METHOD

(71) Applicants: Junji Kajihara, Tokyo (JP); Shinichi Kai, Tokyo (JP); Isao Ichimura, Tokyo (JP); Yuji Segawa, Tokyo (JP)

(72) Inventors: Junji Kajihara, Tokyo (JP); Shinichi Kai, Tokyo (JP); Isao Ichimura, Tokyo (JP); Yuji Segawa, Tokyo (JP)

(73) Assignee: Sony Corporation, Tokyo (JP)

(21) Appl. No.: 13/546,733

(22) Filed: Jul. 11, 2012

(30) Foreign Application Priority Data
Aug. 3, 2011 (JP) 2011-169993

(51) Int. Cl. G01N 21/75; C12M 1/40

(52) U.S. Cl. 435/6.12; 422/82.05; 435/287.2; 436/164

(57) ABSTRACT

Disclosed herein is an optical analysis apparatus including: a light source; a light guiding plate configured to guide incident light from the light source to each of reaction areas; a light shielding structure configured to restrict emission directions of light beams emitted from the inside of the reaction areas; and a detection system configured to detect the light beams emitted from the inside of the reaction areas by radiation of the light.

### BACKGROUND

The present disclosure relates to an optical analysis apparatus and an optical analysis method. To put it in more detail, the present disclosure relates to an optical analysis apparatus for, gene expression analyses, infectious-disease examinations, gene analyses such as SNP (single nucleotide polymorphism) analyses, protein analyses and cell analyses, and also relates to an optical analysis method adopted in the optical analysis apparatus.

In recent years, research of technologies related to gene analyses, protein analyses, cell analyses and the like has been making progress in a variety of fields including the medical field, the drug development field, the clinical examination field, the food field, the agricultural field and the industrial field. Most recently in particular, the technological development and practical realization of a lab-on-a-chip have been making progress. In a micro-scale flow path and a micro-scale well provided in such a lab-on-a-chip, a variety of reactions of, for example, detections and analyses of nucleic acids, proteins, cells, and the like are carried out. Serving as a technique to easily measure a biological molecule and the like, such a technology draws much attention.

In this case, in order to be capable of detecting and measuring even an analyte having a very small quantity, in general, a method making use of a nucleic-acid amplification reaction is adopted, for example. The nucleic-acid amplification reaction is based on a PCR (Polymerase Chain Reaction) technique amplifying a DNA (Deoxyribonucleic Acid) fragment by several hundreds of thousands of times.

In addition, an optical analysis apparatus is being developed as an apparatus configured to detect and measure even a small quantity of a desired substance in a number of analytes in an analysis of light such as absorbed light, fluorescent light or emitted light by making use of typically a microplate having a number of wells.

In recent years, an optical analysis apparatus making use of an LED (light emitting diode) or a semiconductor laser as a light source in place of a tungsten halogen lamp or an electrical discharge tube has been becoming a mainstream. There has also been known an absorptiometer having a radiation mechanism in which light emitted by an LED is directly radiated to a sample. (For example, refer to Japanese Patent Laid-open No. Hei 9-264845). In a second embodiment of this absorptiometer, an configuration is exemplified in which a plurality of measured members of an analyte are arranged in a matrix, and, for the matrix, a plurality of LEDs and a plurality of light receiving devices each forming a pair in conjunction with one of the LEDs are included.

However, stray light (crosstalk) is generated with ease in the optical analysis apparatus. Since the crosstalk causes incorrect detections, there is raised a problem that the detection precision of the optical analysis apparatus is lowered.

### SUMMARY

It is thus desirable to provide an optical analysis apparatus having good detection precision and an optical analysis method for the apparatus.

An optical analysis apparatus according to an embodiment of the present disclosure includes: a light source; a light guiding plate configured to guide incident light from the light source to each of reaction areas; a light shielding structure configured to restrict emission directions of light beams emitted from the inside of the reaction areas; and a detection system configured to detect the light beams emitted from the inside of the reaction areas by radiation of the light.

Another embodiment of the present disclosure provides an optical analysis method including: guiding light radiated from a light source to each of reaction areas by making use of a light guiding plate; directing light beams emitted from the inside of the reaction areas to a detection system by way of a light shielding structure configured to restrict emission directions of the light beams; and detecting the light beams by making use of the detection system.

By using the light shielding structure, it is possible to suppress stray light (crosstalk) from the reaction areas that may cause incorrect detection.

The light guiding plate makes on-surface batch excitation/detection possible so that, even in the case of a number of reaction areas, analyses can be carried out with a high degree of precision and work efficiency of the analyses can be improved. In addition, the space can be saved, so the optical analysis apparatus can be made compact. It is also possible to make use of a plurality of light sources for generating light beams having different wavelengths so that the optical analysis apparatus can be developed into an apparatus for multi-color detection.

Thus, in an optical analysis of light such as absorbed light, fluorescent light or emitted light, the precision of the light detection can be improved even for a number of analytes. In addition, even when the quantity of a targeted substance in a reaction area is small, the analysis can be carried out with a high degree of precision.

As the light source, it is preferable to make use of one or more laser light sources and/or one or more LED light sources. A laser light source generates high-output laser beams having a narrow spectrum width so that an excitation filter is not required. Thus, the optical analysis apparatus can be made small in size. In addition, by making use of LED light sources, a plurality of LED light sources can be provided at a low cost. The batch excitation makes the multi-color detection possible.

It is preferable to detect light beams, which are generated from the inside of the reaction areas by radiation of excitation light beams having different wavelengths from the light source, on a time-division basis. Thus, multi-color excitation is possible and fluorescent light beams having different wavelengths can be detected with a high degree of precision.

It is preferable to provide the light shielding structure in such a way that the light shielding structure is brought into contact with a surface of a substrate in which the reaction areas mounted on the optical analysis apparatus are formed.

It is preferable that the light shielding structure has a plurality of apertures configured to restrict the emission directions of the light beams.

It is preferable to provide a plurality of such light shielding structures so as to sandwich a filter.

It is preferable that the optical analysis apparatus is a nucleic-acid amplification reaction apparatus, and that the optical analysis method is a nucleic-acid amplification reaction analysis method. Even a small quantity of analyte can be well analyzed.

The embodiments of the present disclosure provide an optical analysis apparatus having good detection precision and an optical analysis method for the apparatus.

### BRIEF DESCRIPTION OF THE DRAWINGS

FIG. 1 is a cross-sectional diagram showing a typical configuration of an optical analysis apparatus according to a first embodiment of the present disclosure;

FIG. 2 is a cross-sectional diagram showing a typical configuration of an optical analysis apparatus according to a second embodiment of the present disclosure;

FIG. 3 is a cross-sectional diagram showing a typical configuration of an optical analysis apparatus according to a third embodiment of the present disclosure;

FIG. 4 is a cross-sectional diagram showing a typical configuration of an optical analysis apparatus according to a modification example of the present disclosure;

FIG. 5 is a typical diagram showing an angle of light incidence to a detection filter;

FIG. 6 is a diagram showing a typical wavelength of an excitation light generated by a laser light source in the optical analysis apparatus according to the embodiments of the present disclosure;

FIG. 7 is a diagram showing typical wavelengths of different excitation lights generated by different LED light sources in the optical analysis apparatus according to the embodiments of the present disclosure; and

FIG. 8A to 8D is a diagram showing a typical multi-color detection system of the optical analysis apparatus according to the embodiments of the present disclosure.

### DETAILED DESCRIPTION OF THE PREFERRED EMBODIMENTS

Preferred embodiments of the present disclosure are explained by referring to the accompanying diagrams as follows. It is to be noted that each of the embodiments described below is merely a typical implementation of the present disclosure. Thus, each of the embodiments is not to be interpreted as a restriction narrowing the range of the present disclosure.

The present disclosure is described as topics arranged as follows.

### 1: Optical Analysis Apparatus

### 1-1: Light Source

### 1-2: Reaction Areas

### 1-3: Light Shielding Structure

### 1-4: Detection System

### 2: Optical Analysis Apparatus 1 According to First Embodiment

### 3: Optical Analysis Apparatus 1 According to Second Embodiment

### 4: Optical Analysis Apparatus 1 According to Third Embodiment

### 5: Modification Example of Optical Analysis Apparatus 1

### 1: Optical Analysis Apparatus

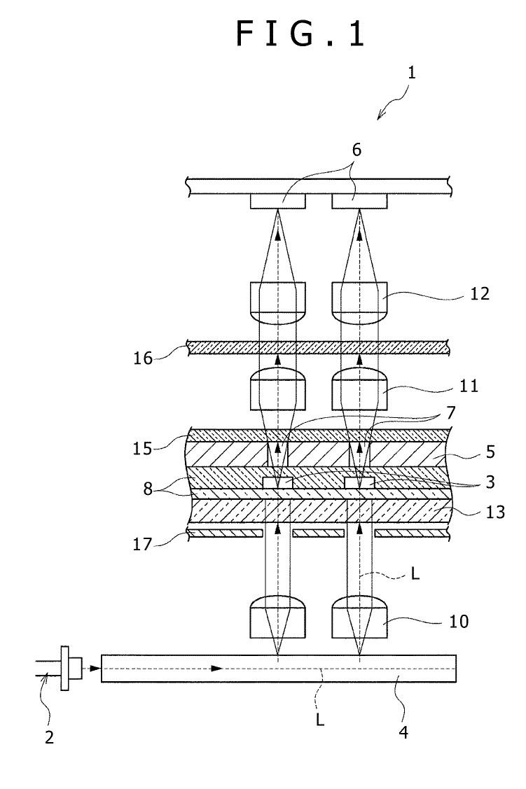

As shown in FIGS. 1 to 4, an optical analysis apparatus 1 according to embodiments of the present disclosure includes a light source 2, a light shielding structure 5 and a detection system 6.

It is proper that the optical analysis apparatus 1 employs a light guiding plate 4 configured to guide incident light radiated from the light source 2 or a plurality of light sources 2 to each of reaction areas 3. It is also proper that the light shielding structure 5 restricts an emission direction of light emitted from the inside of the reaction areas 3. It is also proper that the detection system 6 detects light emitted from the inside of the reaction areas 3 by excitation light. In addition, the reaction areas 3 can be mounted on and dismounted from the optical analysis apparatus 1.

### 1-1: Light Source

The number of the light sources 2 may be singular or plural. By making use of the plurality of light sources 2, it is possible to construct a multi-color light source so that, as a result, multi-color excitation is possible. Thus, an optical analysis for different wavelengths can be carried out. Detection on a time-division basis is also possible. It is to be noted that radiation timings of the light source 2 or the plurality of light sources 2 as well as outputs including a wavelength of excitation light and the quantity of the excitation light can be controlled by a control part.

The light source 2 can have any arbitrary shape and can be provided at any arbitrary location as long as the shape and the location allow light emitted from the light source 2 to be radiated to the reaction areas 3.

In addition, it is preferable to provide the light guiding plate 4 configured to guide incident light from the light source 2 to each of the reaction areas 3. The light guiding plate 4 has an incident-light receiving section or a plurality of incident-light receiving sections at, for example, an edge of the light guiding plate 4. Light emitted from the light source 2 or the light sources 2 is radiated to the incident-light receiving section or the incident-light receiving sections. A member configured to guide the incident light to each of the reaction areas 3 such as a prism, a reflection plate, an unevenness member or the like is provided inside the light guiding plate 4.

By the way, by making use of a light guiding plate, it is possible to carry out on-surface batch excitation for numerous reaction areas. Thus, the number of components can be reduced. An optical analysis apparatus can be made compact, thin and light. In addition, uniform light can be radiated to each of the reaction areas. In the past, it has been necessary to provide a plurality of light sources each corresponding to one of the reaction areas. By making use of the light guiding plate, however, it is possible to carry out on-surface batch excitation even in the case of fewer light sources. It is thus possible to carry out developments toward multi-color detection.

In addition, since the optical analysis apparatus employs the light guiding plate, by making use of various types of light sources, multi-color excitation can be carried out. Multi-color merits are explained as follows. An internal standard can be adopted for each of the reaction areas so that the precision of the optical analysis can be improved. If a reaction is carried out in each of the reaction areas, the speed of the reaction can be calibrated so that quantitative improvement is possible. The number of detection objects that can be detected on one substrate (or one chip) having a plurality of reaction areas can be increased or, to be more specific, can be doubled (multiplex detection) so that the work efficiency can be improved. A typical example of the detection objects is a disease-causing agent.

It is to be noted that the light source 2 is not prescribed in particular. It is, however, preferable that the light source 2 is capable of emitting desired light according to an analyte which is a substance to be analyzed. Typical examples of the light source 2 include a laser light source, an LED light source, a mercury lamp and a tungsten lamp. One of or a plurality of these light sources may be employed.

In the case of the laser light source, the spectrum width is narrow and the intensity of the output light is high. Thus, it is possible to eliminate an excitation filter which was required in the past. In addition, the utilization of a light guiding plate makes it possible to carry out multi-color excitation by making use of variety types of laser light sources having different emitted-light wavelengths. In this case, time division is also possible.

The LED light sources can be light sources for a red color, an orange color, a yellow color, a green color, a blue color, a white color and an ultraviolet, to mention a few. An LED light source or a combination of plural LED light sources may be employed. As a multi-color LED light source, for example, there are a three-color LED light source, four-color LED light source and the like. By making use of excitation filters, the light emitted by these LED light sources can be utilized as the desired excitation light. In addition, by making use of the light guiding plate, multi-color excitation based on variety types of LED light sources can be carried out. In this case, time division is also possible. In the case of a multi-color LED light source, in addition to the batch excitation, it is also possible to carry out sequential excitation without making use of a light guiding plate.

### 1-2: Reaction Areas

Each of the reaction areas 3 is an area in which a sample to be optically analyzed exists. The reaction area 3 may also be used as a field where a reaction for an optical analysis takes place. As an alternative, the reaction area 3 may also be used as a location to be filled up with a sample after a reaction for the purpose of an optical analysis. Typical examples of the reaction for the optical analysis include reactions for detection of absorbed light, detection of fluorescent light and detection of turbidity. In this case, it is preferable that the reaction area 3 is an area that can be used as a location for a reaction such as a nucleic-acid amplification reaction so that a real-time detection is possible. It is preferable to form the plurality of reaction areas 3 inside a reaction container which can be mounted on the optical analysis apparatus 1.

The reaction areas 3 are formed by making use of a substrate or a plurality of substrates. The substrate can be formed by carrying out processes including a wet etching process, an injection formation process and a cutting process on a glass substrate layer. In addition, the shape of the reaction area 3 can be set properly. For example, the reaction area 3 may have a well shape or a fine-canal shape.

A material of the substrate is not prescribed in particular. Typically, the substrate material can be properly selected by considering factors such as a detection method, easiness of fabrication and durability. As the substrate material, for example, a material having a heat resistance and an optical permeability can be properly selected according to the desired optical analysis. Typical examples of such a substrate material include glass and various kinds of plastics such as polypropylene, polycarbonate, cycloolefin polymer and polydimethyl siloxane.

It is to be noted that the inside of the reaction area 3 may be filled up with a reagent, such as a reagent for a nucleic-acid amplification reaction, appropriate for the desired optical analysis.

### 1-3: Light Shielding Structure

The light shielding structure 5 restricts the emission direction of light coming from the inside of the reaction area 3. It is thus possible to suppress stray light generated from a neighboring reaction area, particularly from an adjacent reaction area, the stray light serving as a cause of incorrect detection. By suppressing such stray light, the detection precision can be improved. It is preferable that the light shielding structure 5 has an aperture 7 with a predetermined shape or a plurality of such apertures 7 and that the light shielding structure 5 has a form of a plate with a predetermined thickness.

It is preferable to provide each of the apertures 7 in an area facing one of the reaction areas 3.

In order to restrict the emission direction of light coming from the inside of the reaction area 3, it is preferable that each of the apertures 7 has a predetermined opening shape and depth. By adjusting the opening shape and the depth, it is possible to restrict the emission direction of light coming from the inside of the reaction area 3. By adjusting the opening shape and the depth for example, it is also possible to adjust an incidence angle formed by a detection filter in conjunction with light incident to the detection filter. Since the incidence angle of light can be adjusted in this way, it is also possible to cope with a variety of detection filters by adjusting the aperture portion.

As shown in FIG. 5, if the detection filter is an interference filter (dielectric multi-layer film) for example, it is preferable that the aperture 7 has its width and depth adjusted so that an incidence angle θ with regard to the filter is small when light passes through the inside of the aperture. In the figure, a reference character “a” denotes the width (length of a longitudinal side or a lateral side, or diameter) of the shape of the inside of the aperture whereas a reference character “b” denotes the depth of the shape of the inside of the aperture. By making the width “a” small and the depth “b” large, it is possible to suppress stray light even better. A small incidence angle θ is preferred because, the smaller the incidence angle θ is, the larger the amount of stray light that can be suppressed becomes. Concretely, it is preferable that the incidence angle θ has a value in the following range: θ<20°. By having such an incidence angle θ, the SN (Signal to Noise) ratio can be increased.

As another example, if the detection filter is an absorption filter, it is preferable that the aperture 7 has its width and depth adjusted so that the incidence angle θ with regard to the filter is large when light passes through the inside of the aperture. The reference character “a” denotes the width (length of a longitudinal side or a lateral side, or diameter) of the shape of the inside of the aperture whereas the reference character “b” denotes the depth of the shape of the inside of the aperture. By making the width “a” large and the depth “b” small, it is possible to lengthen a light propagation distance in the detection filter and thus suppress stray light even better. A large incidence angle θ is preferred because, the larger the incidence angle θ is, the larger the amount of stray light that can be suppressed becomes. Concretely, it is preferable that the incidence angle θ has a value in the following range: 20°<θ<70°. By having such an incidence angle θ, the SN ratio can be increased.

For example, the aperture 7 may have a shape which blocks approximately a center portion of light coming from the reaction area 3 but allows light to pass through a portion surrounding the center portion. As an example, a light shielding member (having a disk shape, for example) not shown in the figure may be provided at a position corresponding to the approximately center portion of light coming from the reaction area 3. A bridge member also not shown in the figure may be provided so as to serve as a bridge between the light shielding member and the aperture 7.

In addition, it is only necessary to properly provide components such as a light converging lens and a reflection plate so that light passing through the detection filter is guided into the detection system 6.

An aperture shape is not limited to a circular shape. The aperture shape can be rectangular or polygonal. It is preferable that a surface of the aperture shape is provided approximately in parallel to the reaction area 3.

A tridimensional shape of the aperture 7 may be a cylindrical shape, a rectangular-column shape, a polyhedral shape or the like. For example, the inside of the aperture 7 may be tapered.

From a cost point of view, it is preferable that the aperture 7 has a portion (such as a hole), penetrating the light shielding structure 5, formed in an area facing the reaction area 3 or a plurality of such portions, penetrating the light shielding structure 5, each formed in an area facing one of the reaction areas 3.

The light shielding structure 5 can be constructed by forming the aperture 7 or the plurality of apertures 7 in a pattern formation process carried out by performing an etching process adopting a photolithography method, for example, on a metallic film made of a material such as stainless steel, copper (Cu) or nickel (Ni).

It is only necessary to provide the light shielding structure 5 on at least one of an excitation light incidence side and a fluorescent-light emission side. In this case, it is preferable that the light shielding structure 5 is provided in such a way that the light shielding structure 5 can be brought into contact with a surface of a substrate 8 mounted in the optical analysis apparatus 1 as a substrate in which the reaction areas 3 are formed. It is thus possible to better lower the degree of intrusion of stray light from neighboring reaction areas.

In addition, it is also possible to provide a configuration in which an optical filter such as an excitation filter or a detection filter is clipped and held by a plurality of light shielding structures. With such a configuration, it is possible to limit a light-beam angle at which a light beam passes through the optical filter. It is also possible to effectively extract only a desired wavelength component.

In addition, the light shielding structure 5 may be made mountable on and dismountable from the optical analysis apparatus 1 by adoption of a sliding technique or the like. Thus, the light shielding structure 5 can be properly replaced with another light shielding structure having an aperture shape and a depth, which are determined in advance, in accordance with the type of the detection filter, that is, in accordance with whether the detection filter is an interference or absorption filter. It is to be noted that a plurality of light shielding structures can be used by superposing them on one another so as to allow the incidence angle of light to be adjusted.

In the past, in order to avoid incorrect detection caused by stray light, excitation and light detection were carried out on each of the reaction areas on a time-division basis. It was thus necessary to provide a light source and a detector for each of the reaction areas. In addition, since the time it takes to carry out one detection cycle was proportional to the number of the reaction areas, there was raised a throughput problem for a case in which a number of analytes were to be measured, for example a case in which a plate having 96 holes is used.

By adoption of the light shielding structure described above, however, it is possible to suppress stray light from neighboring reaction areas. It is also possible to carry out the excitation and the light detection, which were carried out on a time-division basis in the past, as a batch operation. By making use of the light guiding plate, it is possible to carry out on-surface batch excitation so that the detection can be performed by utilizing uniform light. In addition, the time it takes to carry out the detection on a plurality of reaction areas can be shortened substantially.

### 1-4: Detection System

It is only necessary to provide the detection system 6 in such a way that the detection system 6 is capable of detecting light components generated inside the reaction areas 3. The light components typically include transmitted light, fluorescent light and scattered light.

It is also only necessary to provide the detection system 6 with a light detector capable of detecting a desired light component. Typical examples of the light detector are a fluorescent-light detector, a turbidity detector, a scattered-light detector and an ultraviolet-visible spectroscopic detector, to mention a few. The detector can be for example any one of an area imaging device such as a CCD (Charge Coupled Device) and a CMOS (Complementary Metal Oxide Semiconductor) device, a PMT (Photomultiplier Tube), a photodiode and a compact sensor, to mention a few.

It is to be noted that a plurality of fluorescent pigments, which are each excited at a specific one of different wavelengths in one of the reaction areas, each emit fluorescent light at the respective wavelength. In order to detect these light components with a high degree of efficiency, for example, it is possible to employ a multi-band-pass filter which has a transmission band corresponding to a plurality of fluorescent spectra. Then, a plurality of excitation light beams having wavelengths different from each other are respectively radiated on a time-division basis and, synchronously with the radiations of the light beams, the light detector is capable of detecting the intensity of each of the fluorescent light beams.

In addition, the optical analysis apparatus 1 according to the embodiments of the present disclosure may include other components such as light converging lenses 10, 11 and 12, a support base 13, an excitation filter 14, detection filters 15 and 16, a diaphragm 17, support bodies each supporting the respective components and installing the reaction areas 3, and a temperature control part such as a heater. Each of these components can be singular or plural as appropriate. In addition, the optical analysis apparatus 1 may also be provided with a control part configured to control an emission timing of excitation light, an output (a wavelength of the excitation light, an intensity of the excitation light, and the like), a time division and a multi-color time division, to mention a few, thereby controlling each of the above-described components.

The excitation filter 14 can be any proper filter as long as the filter is capable of generating a light component having a specific wavelength desired in accordance with a variety of light analysis methods.

Each of the detection filters 15 and 16 can be any filter as long as the filter is proper for light components required in the detection. The light components required in the detection include fluorescent light, scattered light and transmitted light.

In accordance with requirements, the optical analysis apparatus 1 may be provided with one excitation filter described above and one detection filter described above or a plurality of such excitation filters and a plurality of such detection filters. In some cases, the optical analysis apparatus 1 may not be provided with such an excitation filter or such a detection filter. It is thus possible to generate necessary light components and eliminate unnecessary light components. In addition, it is possible to improve the detection sensitivity of the optical analysis apparatus 1 and the detection precision thereof.

On top of that, each of the detection filters 15 and 16 can be a multi-band-pass filter which has a transmission band corresponding to each fluorescent spectrum. In this case, by driving the light source to emit light on a time-division basis, fluorescent light can be detected. If a detection method making use of two fluorescent pigments having different types is adopted for example, the multi-band-pass filter allows two types of fluorescent-light detection to be carried out. In this case, the multi-band-pass filter is referred to as a dual-band-pass filter.

The aforementioned temperature control part is not prescribed in particular. Typical examples of the temperature control part include a transparent conductive film such as an ITO (Indium Tin Oxide) heater exhibiting light permeability. It is preferable to provide the temperature control part at a position which allows the temperature of the reaction area 3 to be controlled thereby. It is desirable to provide the temperature control part at a position close to the substrate of the reaction area 3 and, in addition, it is preferable to provide the temperature control part in the light emission direction and/or the light incidence direction. In order to make the optical analysis apparatus 1 small in size, it is preferable to make use of the temperature control part also as the support base 13. It is therefore possible to control the temperature of the analyte in each of the reaction areas 3. Thus, a stable detection result can be obtained and the detection precision can be improved. In addition, since the reaction in the reaction area 3 can be controlled, it is possible to detect an analyte (a nucleic-acid amplification reaction, for example) requiring the reaction at a detection time. Thus, the optical analysis apparatus 1 can be used also as a reaction apparatus and an apparatus capable of carrying out an optical analysis and a reaction. Typical examples of the apparatus capable of carrying out an optical analysis and a reaction include a nucleic-acid amplification reaction apparatus.

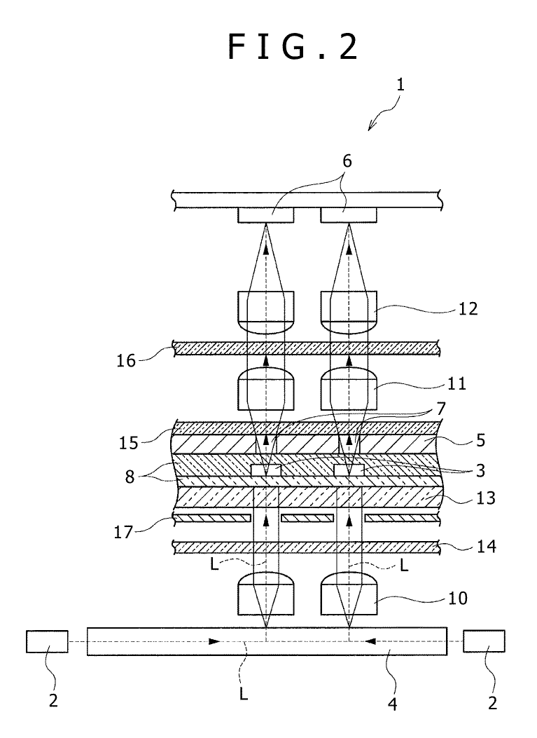

By referring to FIGS. 1 and 2, the following description explains operations carried out by the optical analysis apparatus 1 according to the present disclosure.

Light L from the light source 2 is radiated to the reaction areas 3 which each include an analyte. At that time, the light L can also be radiated to the reaction areas 3 by making use of the light guiding plate 4. The excitation light L is then radiated to the reaction areas 3.

Light components L generated from the inside of the reaction areas 3 by the radiation of the light L to the reaction areas 3 pass through the apertures 7 each having a shape determined in advance and each provided at a location facing one of the reaction areas 3. In this case, the light components L include fluorescent light, transmitted light and scattered light. In this way, since the light components L pass through their respective apertures 7 provided in the light shielding structure 5, the emission directions of the light components L are restricted. It is thus possible to suppress stray light coming from surrounding reaction areas (especially from adjacent reaction areas) to serve as a cause of incorrect detection. Then, the light component L having a restricted emission direction passes through the detection filter 15, the light converging lens 11, the detection filter 16 and the light converging lens 12, becoming a desired light component L. The light component L is detected by the light detector employed in the detection system 6. At that time, stray light coming from surrounding reaction areas is suppressed. Thus, the detection precision of analytes each provided in one of the reaction areas is improved. At a measurement time, the reaction area 3 is used as a reaction field. Thus, the light component L can be detected on a real-time basis. Since the reaction and the detection can be carried out in a row, the optical analysis apparatus 1 offers excellent convenience.

If the light source 2 is a laser light source, it is not necessary to make use of an excitation filter. In this configuration, the excitation light is radiated to the reaction areas 3 (refer to FIG. 1). If the light source 2 is an LED or the like, the excitation light is radiated to the reaction areas 3 by way of the excitation filter 14.

In addition, the excitation filter 14 can also be a multi-band-pass filter. The multi-band-pass filter enables a plurality of different excitation light beams to be radiated to the reaction areas 3. In this case, as for each of the detection filters, it is only necessary to properly make use of a corresponding multi-band-pass filter. It is thus possible to carry out a plurality of optical analyses and detect light components on a time-division basis.

If the light guiding plate 4 is used, it is possible to guide incident light emitted by the light source 2 or a plurality of light sources 2 to the reaction areas 3.

In addition, it is only necessary that the numbers and the types of excitation filters, detection filters and light converging lenses are each determined appropriately in accordance with requirements. That is to say, the numbers and the types are not limited to the above description.

### 2: Optical Analysis Apparatus 1 According to First Embodiment

As shown in FIG. 1, for example, a first embodiment implementing the optical analysis apparatus 1 provided by the present disclosure includes the laser light source 2, the light shielding structure 5 and the detection system 6. In the following description, the first embodiment implementing the optical analysis apparatus 1 provided by the present disclosure is also referred to simply as the optical analysis apparatus 1 according to the first embodiment. Each configuration already described so far is not explained again in the following description.

It is preferable that the optical analysis apparatus 1 according to the first embodiment has the light guiding plate 4 configured to guide incident light emitted by the laser light source 2 or a plurality of laser light sources 2 to the reaction areas 3. It is also preferable that the laser light source 2 or the laser light sources 2 are provided lateral to the light guiding plate 4 or on a side surface of the light guiding plate 4.

It is preferable to properly provide the light converging lenses 10 between the light guiding plate 4 and the substrate 8 in which the reaction areas 3 employed in the optical analysis apparatus 1 are formed. It is also desirable to properly provide the light converging lens and the detection filter or a plurality of light converging lenses and a plurality of detection filters between the light shielding structure 5 and the detection system 6.

In addition, it is desirable that the light shielding structure 5 is provided in such a way that the light shielding structure 5 can be brought into contact with the surface of the substrate 8 in which the reaction areas 3 employed in the optical analysis apparatus 1 are formed. It is also desirable that the light shielding structure 5 is provided in such a way that the light shielding structure 5 can be brought into contact with the detection filter 15. It is also desirable that the light shielding structure 5 is provided so as to be sandwiched between the substrate 8 and the detection filter 15. It is to be noted that the light shielding structure 5 may be provided at a plurality of locations such as a location between the light guiding plate 4 and the substrate 8 as well as a location between the detection filter 15 and the detection system 6. In addition, a plurality of light shielding structures may be used as is the case with a modification example which will be described later.

It is to be noted that each of the light source 2 and the detection system 6 may be supported by a proper support body.

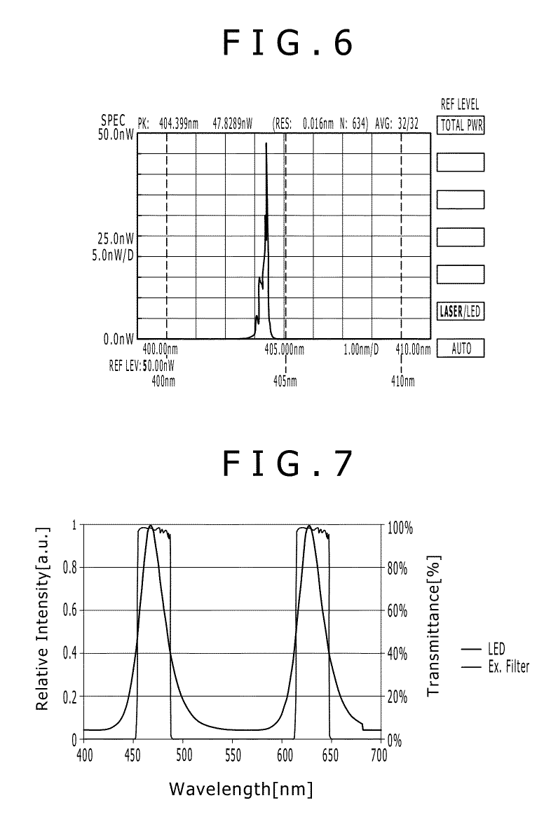

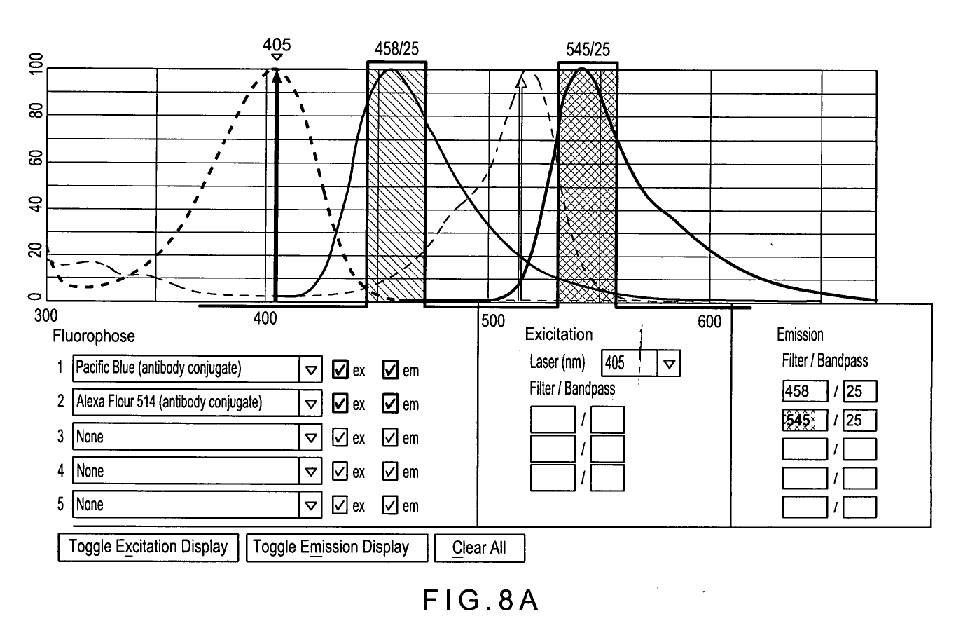

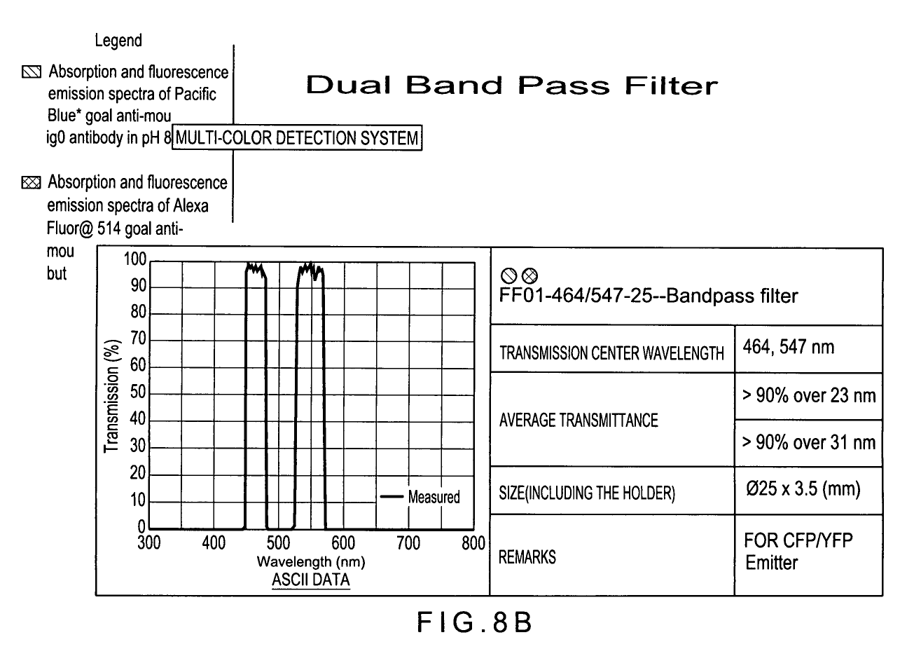

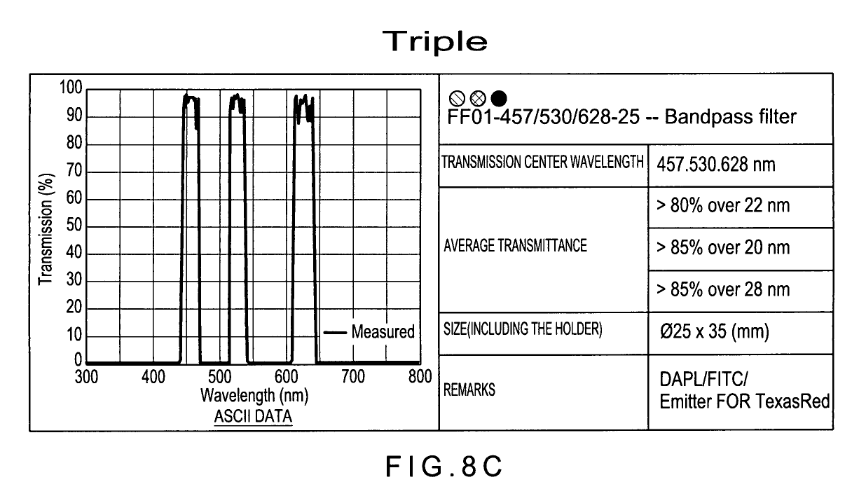

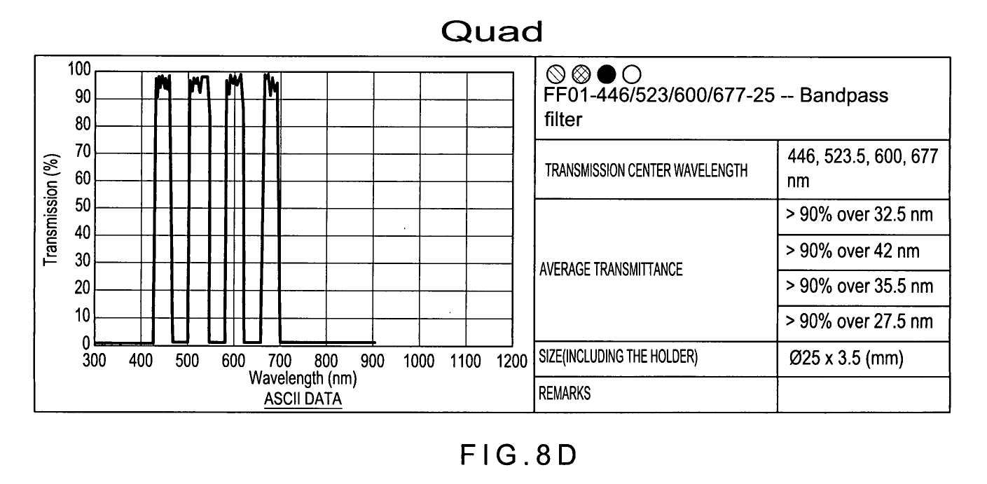

Since the laser used as the light source 2 in the optical analysis apparatus 1 according to the first embodiment has a narrow spectrum width and a high output (refer to FIG. 6), the excitation filter 14 can be arbitrarily eliminated (refer to FIG. 1). It is possible to provide an optical filter in order to remove unnecessary light components.

To put it concretely, the laser generally has a narrower line width in a range of half the spectrum width up to several nanometers compared with an LED having a range of half the spectrum width from several to ten nanometers. Thus, it is not necessary to provide an excitation filter in an excitation system. In addition, with a light guiding plate employed, the use of lasers of a variety of types having different oscillation wavelengths makes multi-color excitation possible. On top of that, it is also possible to carry out on-surface batch excitation and multi-color detection on a time-division basis. In this way, it is possible to make use of a plurality of light sources having different wavelengths even though such light sources were difficult to implement in an installation space provided for light sources in the past. Thus, the detection precision can be improved and the work efficiency can be increased. In addition, the optical analysis apparatus 1 can be made more compact.

By referring to FIG. 1, the following description explains typical operations carried out by the optical analysis apparatus 1 according to the first embodiment of the present disclosure.

A laser beam L (excitation light having a specific wavelength) is emitted by the light source 2. The excitation light L propagates to the incident-light receiving section of the light guiding plate 4. The incident excitation light L passes through the light guiding plate 4 and becomes a plurality of substantially uniform excitation light beams L. At about the same time, the excitation light beams L are guided to their respective reaction areas 3. The excitation light beams passing through the light guiding plate 4 are radiated to their respective reaction areas 3 in the substrate 8 by way of the diaphragm 17 and the support base 13. The radiation causes light components (such as fluorescent light) to be generated from the inside of the reaction areas 3. The light components pass through their respective apertures 7 which are provided in the light shielding structure 5 at positions facing their respective reaction areas 3. Thus, the emission directions of the light from the inside of the reaction areas 3 are restricted so that it is possible to suppress stray light coming from neighboring reaction areas. The light components propagate to the detection system 6 by way of the detection filters 15 and 16, being detected by the light detector employed in the detection system 6.

### 3: Optical Analysis Apparatus 1 According to Second Embodiment

As shown in FIG. 2, for example, a second embodiment implementing the optical analysis apparatus 1 provided by the present disclosure includes the LED light source 2, the excitation filter 14, the light shielding structure 5 and the detection system 6. In the following description, the second embodiment implementing the optical analysis apparatus 1 provided by the present disclosure is also referred to simply as the optical analysis apparatus 1 according to the second embodiment. Each configuration already described so far is not explained again in the following description.

It is preferable that the optical analysis apparatus 1 according to the second embodiment has the light guiding plate 4 configured to guide incident light L emitted by the LED light source 2 or a plurality of LED light sources 2 to the reaction areas 3. It is also preferable that the LED light source 2 or the LED light sources 2 are provided lateral to the light guiding plate 4 or on a side surface of the light guiding plate 4.

It is only necessary that light emitted by the LED light source 2 becomes excitation light having a desired specific wavelength before the emitted light is radiated to the reaction areas 3.

It is only necessary that the excitation filter 14 configured to convert the LED light into the excitation light having a desired specific wavelength is provided at a position between the light source 2 and the reaction areas 3. For example, the excitation filter 14 may be provided at a position between the LED light source 2 and the incident-light receiving section of the light guiding plate 4 or between the light guiding plate 4 and the reaction areas 3.

If the excitation filter 14 is provided at a position between the LED light source 2 and the incident-light receiving section of the light guiding plate 4, the excitation filter 14 may convert the light emitted by the LED light source 2 into the excitation light having a specific wavelength and the excitation light may be then supplied to the light guiding plate 4. As an alternative to this configuration, the excitation filter 14 may be provided on the incident-light receiving section.

If the excitation filter 14 is provided at a position between the light guiding plate 4 and the reaction areas 3, it is more preferable to provide the excitation filter 14 at a position between the light guiding plate 4 and the substrate 8 in which the reaction areas 3 employed by the optical analysis apparatus 1 are formed.

In addition, it is desirable that the excitation filter 14 is a multi-band-pass filter. If a plurality of LED light sources 2 are used and the excitation filter 14 is a multi-band-pass filter, multi-color excitation can be carried out. Thus, multi-color detection can also be carried out as well. On top of that, a plurality of detections (such as a plurality of fluorescent components) can be carried out by adoption of a time-division technique.

It is to be noted that, since the light converging lens, the detection filter and the light shielding structure have been explained in the description of the optical analysis apparatus 1 and the description of the first embodiment, these descriptions are omitted here. In addition, a plurality of light shielding structures may be used as is the case with a modification example which will be described later.

By referring to FIG. 2, the following description explains typical operations carried out by the optical analysis apparatus 1 according to the second embodiment of the present disclosure.

Light L is emitted by the LED light source 2. The light L propagates to the incident-light receiving section of the light guiding plate 4 and is then subjected to on-surface batch radiation so that the light L is guided to the reaction areas 3 by way of the light guiding plate 4. The radiated light L is converted by the excitation filter 14 into excitation light L having a specific wavelength. The excitation light L is radiated to the respective reaction areas 3 in the substrate 8 by way of the respective light converging lenses 10, the diaphragm 17 and the support base 13. The radiation causes light components L such as fluorescent light to be generated from the inside of the reaction areas 3. The light components L pass through the respective apertures 7 which are provided on the light shielding structure 5 at positions facing their respective reaction areas 3. Thus, the emission directions of the light from the inside of the reaction areas 3 are restricted so that it is possible to suppress stray light coming from neighboring reaction areas or, to be more specific, from adjacent reaction areas. The light components L propagate to the detection system 6 by way of the detection filter 15, the light converging lens 11, the detection filter 16 and the light converging lens 12, being detected by the light detector employed in the detection system 6.

If the excitation filter 14 is a multi-band-pass filter, then it is also possible to make use of a plurality of LED light sources 2 and drive the LED light sources 2 to emit light on a time-division basis (refer to FIG. 7). For example, while one of the light sources is emitting light, no other one of the light sources emits light. To put it more concretely, while excitation light having a wavelength of approximately 450 nm is being radiated and then detected, excitation light having a wavelength of approximately 620 nm is not radiated. While excitation light having a wavelength of approximately 620 nm is being radiated and then detected, excitation light having a wavelength of approximately 450 nm is not radiated. These typical operations are carried out repeatedly and alternately.

In the case of a plurality of colors, it is thus possible to carry out detection for one color at a time so as to cope with substances reacting to a plurality of light beams in the reaction areas 3. For example, the substances are a plurality of fluorescent components. As a result, it is possible to improve the work efficiency and the detection precision.

### 4: Optical Analysis Apparatus 1 According to Third Embodiment

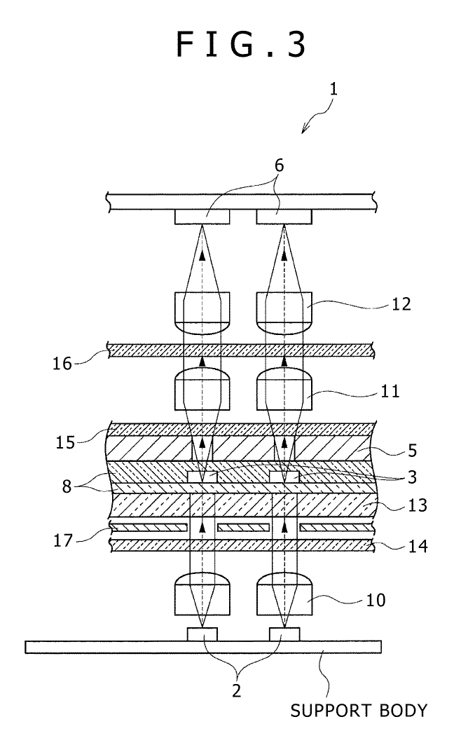

As shown in FIG. 3, for example, a third embodiment implementing the optical analysis apparatus 1 provided by the present disclosure includes the LED light source 2, the excitation filter 14, the light shielding structure 5 and the detection system 6. In the following description, the third embodiment implementing the optical analysis apparatus 1 provided by the present disclosure is also referred to simply as the optical analysis apparatus 1 according to the third embodiment. Each configuration already described so far is not explained again in the following description.

The optical analysis apparatus 1 according to the third embodiment employs a plurality of LED light sources 2 each provided for one of the reaction areas 3. Thus, light can be radiated to the reaction areas 3 in a batch operation. In addition, it is desirable that each of the LED light sources 2 functions as a multi-color LED. By employing the multi-color LED along with an excitation filter serving as a multi-band-path filter, excitation operations can be carried out sequentially so that detection can be executed on a time-division basis. It is to be noted that, by making use of a light guiding plate, the number of LED light sources can be reduced.

It is desirable that light emitted by the LED light source 2 becomes excitation light having a desired specific wavelength before the emitted light is radiated to the reaction areas 3. It is preferable that the excitation filter 14 is provided at a position between the LED light sources 2 and the substrate 8 on which the reaction areas 3 employed by the optical analysis apparatus 1 are formed.

It is to be noted that, since the light converging lens, the detection filter and the light shielding structure have been explained in the description of the optical analysis apparatus 1, the description of the first and second embodiments, these descriptions are omitted here. In addition, a plurality of light shielding structures may be used as is the case with a modification example which will be described later.

By referring to FIG. 3, the following description explains typical operations carried out by the optical analysis apparatus 1 according to the third embodiment of the present disclosure.

Light L is emitted by the LED light sources 2 at the same time so that the light L can be radiated to the reaction areas 3 in on-surface batch radiation. The excitation filter 14 converts the light L emitted by each of the LED light sources 2 into the excitation light L having a specific wavelength. The excitation light L passes through the diaphragm 17 and the support base 13, being radiated to the reaction areas 3 in the substrate 8. The radiation causes light components L such as fluorescent light to be generated from the inside of the reaction areas 3. The light components L pass through the respective apertures 7 which are provided on the light shielding structure 5 at positions facing the respective reaction areas 3. Thus, the emission directions of the light from the inside of the reaction areas 3 are restricted so that it is possible to suppress stray light coming from neighboring reaction areas. The light components L propagate to the detection system 6 by way of the detection filter 15, the light converging lens 11, the detection filter 16 and the light converging lens 12, being detected by the light detector employed in the detection system 6.

In addition, if the excitation filter 14 is a multi-band-pass filter, it is also possible to make use of the plurality of LED light sources 2 and drive the LED light sources 2 to emit light on a time-division basis. In the case of a plurality of colors, it is thus possible to carry out detection for one color at a time so as to cope with a plurality of fluorescent components in the reaction areas 3. As a result, it is possible to improve the work efficiency and the detection precision.

### 5: Modification Example of Optical Analysis Apparatus 1

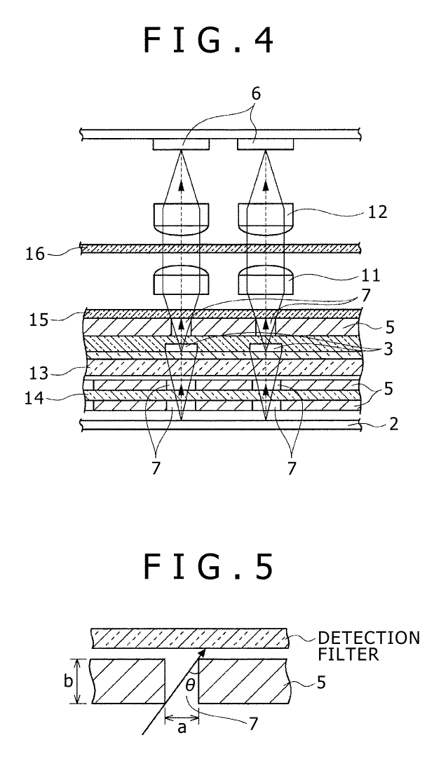

A modification example of the optical analysis apparatus 1 according to the present disclosure includes the light source 2, the excitation filter 14, a plurality of light shielding structures 5 and the detection system 6 as shown in FIG. 4, for example. In the following description, the modification example of the optical analysis apparatus 1 according to the present disclosure is also referred to simply as the optical analysis apparatus 1 according to the modification example. Each configuration already described so far is not explained again in the following description.

In the optical analysis apparatus 1 according to the modification example, an optical filter is clipped and held by the plurality of light shielding structures 5. Thus, even if no light converging lenses are provided between the reaction areas 3 and the light source 2, light can be radiated to the reaction areas 3 in on-surface batch radiation. In addition, the optical analysis apparatus 1 according to the modification example can be made smaller in size. Since stray light can also be suppressed, the detection precision can be improved as well. It is to be noted that the optical analysis apparatus 1 according to the first to third embodiments may adopt the same configuration as this modification example or may incorporate this configuration provided that the incorporation of the configuration is within a range not losing the effects provided by the present disclosure.

The optical analysis apparatus 1 according to the embodiments of the present disclosure is capable of carrying out a variety of optical analyses such as nucleic-acid amplification detection and metal detection. The optical analysis apparatus 1 is also capable of carrying out the analyses on a real-time basis. In addition, when the optical analysis apparatus 1 is provided with a temperature control part configured to control the temperature, the optical analysis apparatus 1 is capable of functioning also as a reaction apparatus. Typical examples of the reaction apparatus include a nucleic-acid amplification reaction apparatus. As an example, a nucleic-acid amplification reaction is explained as follows.

### [Nucleic-Acid Amplification Reaction]

The nucleic-acid amplification reaction according to the present disclosure includes the related-art PCR (Polymerase Chain Reaction) method implementing temperature cycling and a variety of isothermal amplification methods not implementing the temperature cycling. Typical isothermal amplification methods include a LAMP (Loop-Mediated Isothermal Amplification) method, a SMAP (SMart Amplification Process) method, a NASBA (Nucleic Acid Sequence-Based Amplification) method, an ICAN (Isothermal and Chimeric primer-initiated Amplification of Nucleic acids) method (a registered trademark), a TRC (Transcription-Reverse transcription Concerted) method, an SDA (Strand Displacement Amplification) method, a TMA (Transcription-Mediated Amplification) method and an RCA (Rolling Circle Amplification) method, to mention a few.

In addition, the vast majority of nucleic-acid amplification reactions are nucleic-acid amplification reactions carried out at a variable or constant temperature for the purpose of amplifying a nucleic acid. In addition, these nucleic-acid amplification reactions also include a reaction accompanying quantitative estimation of an amplified nucleic-acid chain such as a RT-PCR (Real-Time PCR) method and an RT-LAMP method.

In addition, reagents include a reagent required for obtaining an amplified nucleic acid chain in the nucleic-acid amplification reaction described above. To put it concretely, the reagents include an oligonucleotide primer, a nucleic-acid monomer (dNTP), an enzyme and a reaction buffering solution which have been formed into a base sequence complementary to the target nucleic-acid chain.

In the PCR method, the following amplification cycle is carried out continuously: thermal denaturation (at about 95° C.)—primer annealing (at about 55 to 60° C.)→extension reaction (at about 72° C.)

The LAMP method is a method for obtaining dsDNA (double-stranded DNA) as amplified product from DNA and RNA (Ribonucleic Acid) at a constant temperature by utilizing DNA loop formation. As an example, components (i), (ii) and (iii) are added so that an inner primer is capable of forming a stable base paring for a complementary sequence on a template nucleic acid and progression is made by incubation at a temperature at which a chain substitution polymerase is capable of sustaining enzyme activation. At that time, it is preferable that the incubation temperature is in a range of 50 to 70° C. and the incubation time is in a range of approximately one minute to ten hours.

Components (i), (ii) and (iii) are described as follows:

Components (i): two types of inner primer, two types of outer primer in addition, or two types of loop primer in further addition
Components (ii): Chain substitution polymerase
Components (iii): Substrate nucleotide

### [Method for Detecting Nucleic-Acid Amplification (Products)]

Typical examples of the method for detecting nucleic-acid amplification include a method making use of a turbid material, a fluorescent material or a chemical light emitting material.

In addition, typical examples of the method making use of a turbid material include a method making use of a pyrroline acid obtained as a result of a nucleic-acid amplification reaction and a deposited material generated by metallic ions which can be bonded with the pyrroline acid. A metallic ion is a univalent or divalent metallic ion. If the metallic ions are bonded with the pyrroline acid, salts insoluble or hardly soluble in water are formed, which results in a turbid material.

To put it concretely, typical examples of the metallic ion include an alkali metallic ion, an alkaline-earth metallic ion and a divalent transition metallic ion. The alkaline-earth metallic ion includes an ion of magnesium (II), calcium (II) or barium (II). The divalent transition metallic ion includes an ion of zinc (II), lead (II), manganese (II), nickel (II) or iron (II). It is desirable that the metallic ions are one type or more types of ions selected among the alkaline-earth metallic ions, the divalent transition metallic ions and the like. It is even more desirable that the metallic ions are the ions of magnesium (II), manganese (II), nickel (II) and iron (II).

It is preferable that the concentration of the metallic ions used as a dopant is in a range of 0.01 to 100 mM and that the detection wavelength is in a range of 300 to 800 nm.

Typical examples of the method making use of a fluorescent material or a chemical light emitting material include an intercalate method making use of a fluorescent pigment (a derivative) specifically inserted into a double stranded nucleic acid to emit fluorescent light and a labeled-probe method making use of a probe with a fluorescent pigment bonded to a specific oligonucleotide for a nucleic-acid sequence to be amplified.

Typical examples of the labeled-probe method include a hybridization (Hyb) probe method and a hydrolytic degradation (TaqMan) probe method.

The Hyb probe method is a method making use of two different probes designed in advance to be close to each other. One of the probes is a probe labeled with a donor pigment whereas the other probe is a probe labeled with an acceptor pigment. When the two probes are hybridized with a target nucleic acid, the acceptor pigment excited by the donor pigment emits fluorescent light.

The TaqMan probe method is a method making use of a probe labeled in such a way that two pigments are close to each other. The two pigments are a reporter pigment and a quencher pigment. When the probe is hydrolyzed at a nucleic-acid extension time, the reporter pigment and the quencher pigment are separated away from each other and, as the reporter pigment is excited, fluorescent light is emitted.

The fluorescent pigment (derivative) utilized in the method making use of a fluorescent material can be typically any one of SYBR (a registered trademark) Green I, SYBR (the registered trademark) Green II, SYBR (the registered trademark) Gold, OY (Oxazole Yellow), TO (Thiazole Orange), PG (Pico Green, where Pico is a registered trademark) and an ethidium bromide, to mention a few.

An organic compound utilized in the method making use of a chemical light emitting material can be typically any one of luminol, lophine, lucigenin and oxalic acid ester, to mention a few.

### [RT-PCR Apparatus According to Present Disclosure]

The following description explains the optical analysis apparatus 1 provided by the embodiments of the present disclosure to serve as a PCR apparatus (RT-PCR apparatus).

The following description explains a method for detecting a nucleic acid in accordance with a procedure including a step Sp1 (thermal denaturation), a step Sp2 (primer annealing), and a step Sp3 (DNA extension) of the RT-PCR apparatus.

At the thermal denaturation (step Sp1), the temperature control part controls the temperature in the reaction area 3 to 95° C. and the double stranded DNA is subjected to a denaturalization process to be converted into a single stranded DNA.

At the following primer annealing (step Sp2), the temperature in the reaction area 3 is set at 55° C. and a primer is bonded with the single stranded DNA in a complementary base sequence.

Subsequently, at the DNA extension (step Sp3), the temperature in the reaction area 3 is controlled to 72° C. and a cDNA (complementary DNA) is extended by carrying forward a polymerase reaction with the primer as the start point of a DNA synthesis.

By repeating the temperature cycle of such steps Sp1 to Sp3, the DNA in every reaction area 3 is amplified. Fluorescent light generated in the reaction area 3 is detected by the detection system 6 on a real-time basis in order to quantify the amount of the nucleic acid.

In addition, the optical analysis apparatus 1 according to the embodiments of the present disclosure can also be used as a LAMP apparatus (RT-LAMP apparatus).

The temperature in the reaction area 3 is set at a constant value in a range of 60 to 65° C. so as to amplify a nucleic acid in the reaction area 3. It is to be noted that, in accordance with the LAMP method, it is not necessary to carry out the thermal denaturation for converting a double stranded DNA into a single stranded DNA. The primer annealing and the nucleic-acid extension are repeated under the constant-temperature condition.

As a result of the nucleic-acid amplification reaction, a pyrroline acid is generated. Then, metallic ions are bonded with the pyrroline acid in order to produce salts, which are not soluble or hardly soluble in water, as a turbid material (a measurement wavelength in a range of 300 to 800 nm). When incident light is radiated to the turbid material, the incident light becomes scattered light. Then, the detection system 6 measures the quantity of the scattered light on a real-time basis in order to quantify the light. In addition, this quantification can also be carried out from the quantity of the transmitted light.

It is to be noted that the present disclosure can also adopt following configurations:

(1) An optical analysis apparatus including:

a light guiding plate configured to guide incident excitation light from a light source or a plurality of light sources to each of reaction areas;

a light shielding structure configured to restrict emission directions of light beams emitted from the inside of the reaction areas; and

a detection system configured to detect the light beams emitted from the inside of the reaction areas by radiation of the excitation light.

(2) The optical analysis apparatus according to the paragraph (1), in which the light sources radiate light rays having different wavelengths so that the light beams emitted from the inside of the reaction areas can be detected on a time-division basis.

(3) The optical analysis apparatus according to the paragraph (1) or (2), in which the light shielding structure is placed so as to come into contact with a surface of a substrate in which the reaction areas employed by the optical analysis apparatus are formed.

(4) The optical analysis apparatus according to any one of the paragraphs (1) to (3), in which the light shielding structure has a plurality of apertures configured to restrict the emission directions of the light beams.

(5) The optical analysis apparatus according to any one of the paragraphs (1) to (4), in which a plurality of such light shielding structures are provided so as to sandwich a filter.

(6) The optical analysis apparatus according to any one of the paragraphs (1) to (5), the optical analysis apparatus serving as a nucleic-acid amplification reaction apparatus.

(7) An optical analysis method including:

guiding light radiated from a light source or a plurality of light sources to each of reaction areas by making use of a light guiding plate;

directing light beams emitted from the inside of the reaction areas to a detection system by way of a light shielding structure configured to restrict emission directions of the light beams; and

detecting the light beams by making use of the detection system.

(8) The optical analysis method according to the paragraph (7), in which the light sources radiate excitation light rays having different wavelengths so that the light beams emitted from the inside of the reaction areas can be detected on a time-division basis.

(9) The optical analysis method according to the paragraph (7) or (8), in which the light shielding structure is placed so as to come into contact with a surface of a substrate in which the reaction areas employed by an apparatus are formed.

(10) The optical analysis method according to any one of the paragraphs (7) to (9), in which the light shielding structure has a plurality of apertures configured to restrict the emission directions of the light beams.

(11) The optical analysis method according to any one of the paragraphs (7) to (10), in which a plurality of such light shielding structures are provided so as to sandwich a filter so that the emission directions of light are restricted.

(12) The optical analysis method according to any one of the paragraphs (7) to (11), the optical analysis method serving as an optical analysis method for nucleic-acid amplification reactions.

The present disclosure contains subject matter related to that disclosed in Japanese Priority Patent Application JP 2011-169993 filed in the Japan Patent Office on Aug. 3, 2011, the entire content of which is hereby incorporated by reference.

What is claimed is:

1. An optical analysis apparatus comprising:
a light source;
a light guiding plate configured to guide incident light from the light source to each of reaction areas;
a light shielding structure configured to restrict emission directions of light beams emitted from the inside of the reaction areas; and
a detection system configured to detect the light beams emitted from the inside of the reaction areas by radiation of the light.

2. The optical analysis apparatus according to claim 1, wherein the light source is a laser light source.

3. The optical analysis apparatus according to claim 1, wherein the light source is a light emitting diode light source.

4. The optical analysis apparatus according to claim 1, wherein light sources include the laser light source and the light emitting diode light source.

5. The optical analysis apparatus according to claim 1, wherein the light sources radiate light rays having different wavelengths so that the light beams emitted from the inside of the reaction areas can be detected on a time-division basis.

6. The optical analysis apparatus according to claim 1, wherein the light shielding structure is placed so as to come into contact with a surface of a substrate in which the reaction areas employed by the optical analysis apparatus are formed.

7. The optical analysis apparatus according to claim 1, wherein the light shielding structure has a plurality of apertures configured to restrict the emission directions of the light beams.

8. The optical analysis apparatus according to claim 1, wherein the optical analysis apparatus serves as a nucleic-acid amplification reaction apparatus.

9. An optical analysis method comprising:
guiding light radiated from a light source to each of reaction areas by making use of a light guiding plate;
directing light beams emitted from the inside of the reaction areas to a detection system by way of a light shielding structure configured to restrict emission directions of the light beams; and
detecting the light beams by making use of the detection system.
